(19) United States
(12) Patent Application Publication
(10) Pub. No.: US 2005/0019270 A1
(43) Pub. Date: Jan. 27, 2005

(54) Formulation of powder containing nanoparticles for aerosol delivery to the lungs

(76) Inventors: Warren Hugh Finlay, Edmonton (CA); Wilson Roa, Edmonton (CA); Raimar Loebenberg, Edmonton (CA)

Correspondence Address: THOMPSON LAMBERT;SUITE 703D, CRYSTAL PARK TWO, 2121 CRYSTAL DRIVE, ARLINGTON, VA, 22202, US

(21) Appl. No.: 10/624,475

(22) Filed: Jul. 23, 2003

(30) Foreign Application Priority Data
Jul. 21, 2003 (CA) 2,435,632

(51) Int. Cl. A61L 9/04; A61K 9/14

(52) U.S. Cl. 424/46

(57) ABSTRACT

Respirable particles carrying active principles or diagnostics in nanoparticle form are created by mixing the nanoparticles with liquid carrier, then forming the resultant mixture into respirable particles. Spray-drying, freeze spray drying and drying followed by comminution may be used to create the respirable particles, which may be delivered to the lung via a dry powder inhaler. In one example, lactose was used as the excipient and spray-dried with two different types of nanoparticle: gelatin and poly butylcyanoacrylate nanoparticles. The incorporation of nanoparticles did not affect the respirable fraction of the carrier powders.

### BACKGROUND OF THE INVENTION

Aerosols are an effective method to deliver therapeutic agents to the respiratory tract. Nebulizers, metered dose inhalers, or dry powder inhalers are commonly used for this purpose. Local delivery of medication to the lung is highly desirable, especially in patients with specific pulmonary diseases like cystic fibrosis (CF), chronic pulmonary infections or lung cancer. The principal advantages of local delivery include reduced systemic side effects and higher dose levels of the applicable medication at the site of drug action. Unlike the oral route of drug administration, pulmonary inhalation is not subject to first pass metabolism. Therefore, expensive biotechnology drugs like recombinant human deoxyribonuclease (rhDNase) for the treatment of CF or toxic chemotherapeutics are ideal drug candidates for local pulmonary administration. Indeed, aerosol delivery has long been viewed as a promising approach for lung cancer. Given the advantages of pulmonary delivery for certain diseases, it is foreseeable that specialized inhalation treatment for diseases like lung cancer or gene therapy will be developed further.

Carbohydrates and especially mannitol and lactose are widely used as the excipients for dry powder inhalers since they are approved by the Food and Drug Administration (FDA) and other regulatory bodies as excipients for inhalation purposes. This is due to their non-toxic, readily degradable properties after administration. To prepare inhalable powders, spray-drying is a commonly practiced method. In fact, spray-drying has been applied to a variety of substances, such as peptides, antibiotics, vaccines, and carrier particles. One of the principal purposes of aerosolizing spray-dried powders is to achieve powder particle diameters of several micrometers with a narrow particle distribution. This ensures, assuming an appropriate mass median aerodynamic diameter (MMAD), a maximum deposition of the embedded drugs in the tracheo-bronchial and deep alveoli regions for normal inhalation rates.

Independent of the method used to produce an inhalation aerosol, delivery by inhalation must overcome certain obstacles before reaching the site of drug action. This is particularly important when the particle deposition takes place in the upper bronchial area. In this area of the tracheo-bronchial region the epithelium is protected by a mucus layer. Any particle or drug is transported away from the lung by mucociliary clearance. However, the cellular uptake of small molecular weight drugs by epithelium cells or the permeation of such drugs into the systemic circulation is generally expected if the drug can reach the alveolar epithelium, or diffuse through the mucus and reaches the epithelium cells. This might not be the case for large biotechnology molecules like oligonucleotides. Such large molecules might not be able to cross the epithelium or cross the cytoplasm membrane. Consequently they will not be able to reach their site of drug action. This is a general drug delivery problem for large molecules and applies to other routes of administration as well.

Nanoparticles are solid colloidal particles ranging in size from 10 nm to 1000 nm. They can be made from biodegradable and biocompatible biomaterials. Active principles like drugs of oligonucleotides can be adsorbed, encapsulated or covalently attached to their surface or into their matrix. In vitro and in vivo studies have demonstrated that nanoparticles are promising carrier systems for drug targeting strategies.

Studies using inhaled nanoparticles dispersed in aqueous droplets suggest that the mucus clearance can be overcome by nanoparticles, possibly due to rapid displacement of particles to the airway epithelium via surface energetics. Therefore, nanoparticles may be possible vehicles of transporting drugs efficiently to the epithelium, while avoiding unwanted mucociliary clearance.

Cellular uptake studies have demonstrated that besides macrophages, other cells like cancer cells and epithelium cells are also able to take nanoparticles up. Body distribution studies using intravenous injections of nanoparticle preparations have revealed that the surface characteristics of colloidal carriers are one of the most important parameters in avoiding macrophage uptake. Furthermore, in vivo studies have observed an accumulation of nanoparticles in tumor sites. This was attributed to the leaky blood vessel structure of tumors. Such properties make nanoparticles a very attractive delivery vehicle for lung cancer treatment. However, the disadvantage of using nano-sized delivery systems for pulmonary application is that their MMAD is not suitable for inhalation purposes. Due to their size, they reach a transition region where neither diffusion nor sedimentation or impaction are effective deposition mechanisms. Consequently, it is expected that a large fraction of the inhaled dose will be exhaled and little particle deposition will take place in the lung.

The MMAD is the most important parameter regarding pulmonary deposition of particles in the lung. Previous in vivo studies showed that an efficient particle deposition in hamster lungs could be achieved using particles of a size of 6 μm or less in aerodynamic diameter. After particle deposition in the alveoli the particles were submerged in the subphase of the alveolar lining layer and became completely coated with an osmiophilic film. Similar results were observed for particles deposited in the conductive airways. The in vitro experiments showed that the pulmonary surfactant promotes the displacement of particles from the air to the aqueous phase. The extent of the particle immersion depended on the surface tension of the surface-active film. Studies using highly hydrophobic Teflon particles have reported similar results. The surface tension and line tension forces rather than the particles' surface free energy were found to be the decisive force for the displacement of particles into the aqueous phase. Mathematical analysis of the forces involved at the air-fluid interface showed that the surface tension force acting on particles <100 μm was several orders of magnitude greater than forces related to gravity. This means that particles deposited in the peripheral airways and alveoli are submerged in the subphase below the surfactant film and this increases the contact between the epithelial cell and particles. This phenomenon can be utilized for the exposure of nanoparticles to the epithelium cells using fast dissolving carrier particles.

### SUMMARY OF THE INVENTION

The challenge therefore is to design a method for delivering nanoparticles to the lung, to exploit their unique properties in avoiding mucociliary clearance and thus deliver drugs directly to the target tissue or target cells. Such drug delivery may be utilized for therapeutic treatments of lung specific diseases like lung cancer.

There is therefore provided a method of formulating a powder containing nanoparticles for aerosol delivery to the lung. In one aspect of the invention, nanoparticles are mixed with a liquid carrier to create a mixture and the mixture is formed into a powder composed of carrier particles having a size suitable for aerosol delivery to the lung. Such a particle size may be referred to as respirable particle size. The mixture may be formed into a powder by spray drying or by freeze spray drying for example, or by drying followed by milling or other forms of breaking up the mixture. The nanoparticles may contain an active agent for example a drug having a therapeutic, diagnostic or preventative effect on a human being. For aerosol delivery the carrier particles may have a mass median aerodynamic diameter between approximately 1 μm and 5 μm. The carrier particles may be used to deliver nanoparticles to the lung by aerosol delivery. The nanoparticles are incorporated into the matrix of the carrier particles. The MMAD of carrier particles can be adjusted to give sufficient lung deposition in the desired upper or lower generations of the lung e.g. either the bronchial region or the alveolar region. After deposition in the lung the matrix of the carrier particles dissolves and releases the nanoparticles.

Depending on the nature of the nanoparticle matrix there are different functional groups such as carboxyl, sulhydryl and amino groups available for drug binding (covalent or electrostatic). Other biomaterials can also be used to make nanoparticles. The materials can be synthetic, semi-synthetic or natural origin. Active principles can be covalently attached, adsorbed or incorporated to the nanoparticle. The drug loading depends on the functional groups of the biomaterials and on the drug release requirements. Gelatin or other protein based nanoparticles may be incorporated into the carrier particles. Abundant functional groups, such as carboxyl and amino groups, on the particle surface enable easy modification and the covalent binding of drugs. Poly butylcyanoacrylate or other synthetic nanoparticles may be incorporated into the carrier particles.

### BRIEF DESCRIPTION OF THE FIGURES

There will now be described preferred embodiments of the invention with reference to the drawings, in which like reference characters denote like elements, for the purpose of illustrating the invention, and in which:

FIG. 1 is a schematic showing method steps of nanoparticle synthesis with drug loading;

FIG. 2 is a schematic showing method steps of drug loading of nanoparticles using surface modification;

FIG. 3 is a schematic showing synthesis of aerosol particles according to an embodiment of the invention;

FIG. 4 is a schematic showing deposition of nanoparticles in the lung using carrier particles developed according to the invention;

FIGS. 5A, 5B and 5C respectively show a carrier particle with nanoparticles, the carried nanoparticles and the carrier particle matrix;

FIG. 6 is a graph showing comparison of the powder dispersion into an Anderson impactor among different spray-dried powders made according to the invention at different impactor stages; and

FIG. 7 is a graph showing a comparison of the respirable fraction for different powder formulations deagglomerated into an Anderson impactor.

### DETAILED DESCRIPTION OF PREFERRED EMBODIMENTS OF THE INVENTION

In this patent document, the word “comprising” is used in its non-limiting sense to mean that items following the word in the sentence are included and that items not specifically mentioned are not excluded. The use of the indefinite article “a” in the claims before an element means that one of the elements is specified, but does not specifically exclude others of the elements being present, unless the context clearly requires that there be one and only one of the elements.

In a preferred embodiment of the invention, nanoparticles are delivered to the lung via carrier particles that form a powder and which dissolve after coming in contact with the aqueous environment of the lung epithelium. The nanoparticles may be used in drug targeting strategies for pulmonary delivery of drugs and diagnostics. Powder formulation is carried out using two steps, first by mixing nanoparticles with a liquid carrier, and then forming the mixture into a powder of suitably sized carrier particles. Various methods may be used for powder formulation. Respirable particles containing nanoparticles may be created by making droplets out of the liquid and drying the droplets to make a powder. This includes spray drying and freeze-spray drying. The particles may also be made by directly drying the bulk liquid and then breaking up the dried material, for example using comminution, grinding or milling to break up the dried material into respirable particles. The respirable particles can be delivered alone or they can be mixed with other particles to aid in their deagglomeration or manufacturing processing. This includes, for example, lactose carrier particles. Drug may be added in solution to the bulk liquid, thereby incorporating drug in the matrix that holds the nanoparticles.

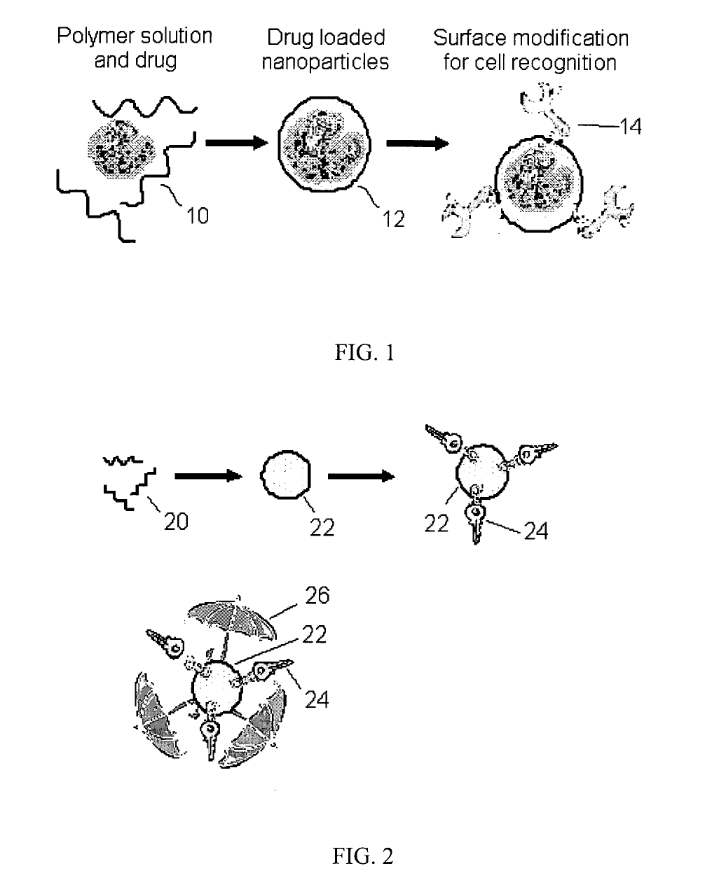

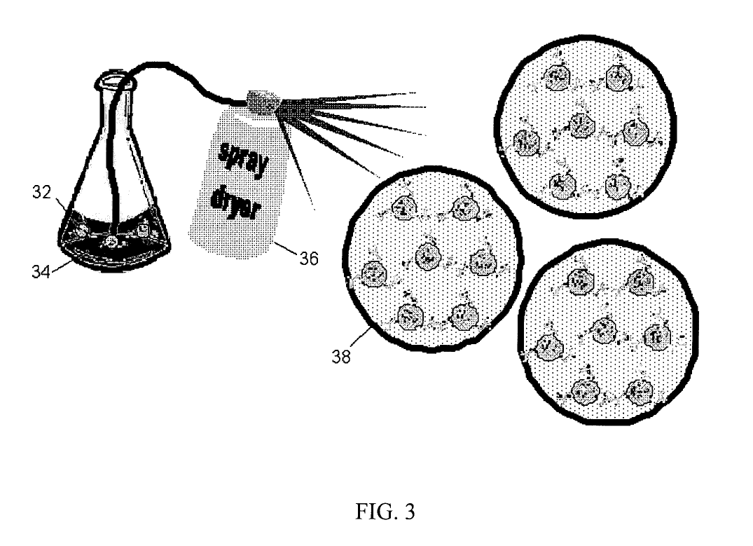

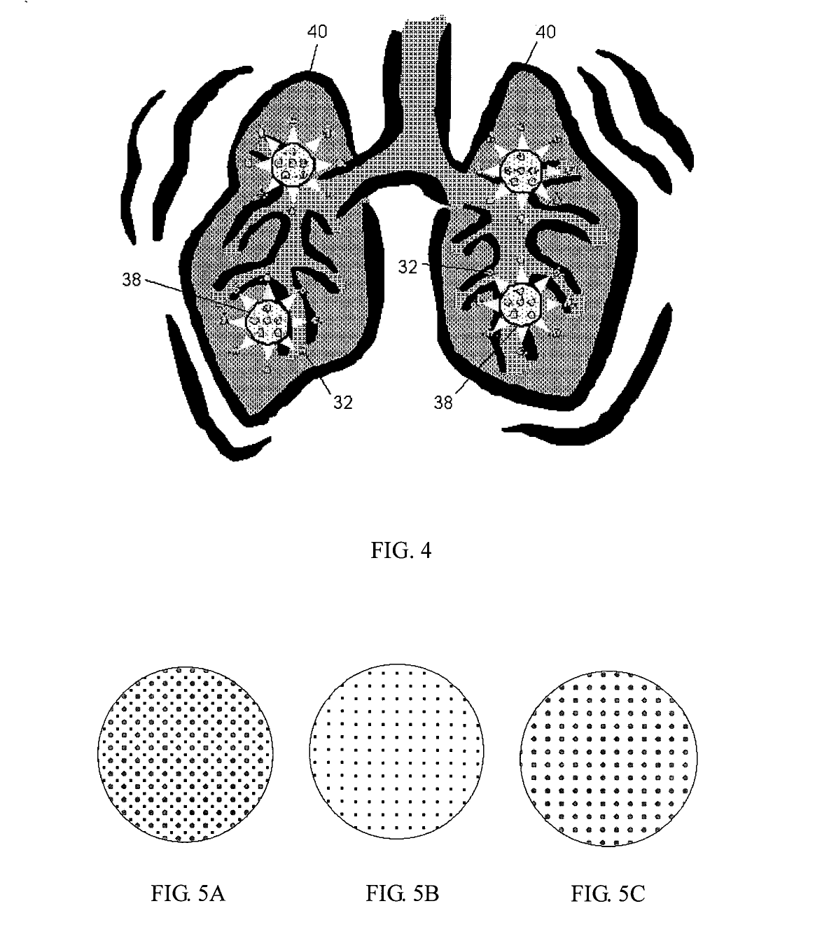

Referring to FIG. 1, a first example of nanoparticle synthesis is illustrated in which drug loaded nanoparticles are formed. First, a solution 10 of a suitable polymer, preferably a macromolecule, and drug is formed, and then in a second step the nanoparticles 12 are obtained from the solution by conventional methods such as by precipitation. Third, the surface 14 of the nanoparticles 12 is modified by conventional methods to achieve a cell-specific uptake. Referring to FIG. 2, an alternative method of nanoparticle synthesis is shown in which a polymer solution 20 is prepared according to conventional principles, followed by formation of nanoparticles 22, such as by precipitation, and attaching of an active principle 24, such as a protein, to the surface of the nanoparticle 22 via S—S bridges or covalent bridges. The nanoparticles 22 may then be surface modified 26 to avoid reticuloendothelial system (RES) uptake or enzymatic degradation. Referring to FIG. 3, nanoparticles 32 are mixed with a carrier liquid 34, such as aqueous lactose or other suitable carrier material and then the resulting mixture is spray dried using a sprayer 36 to form aerosol particles 38 containing nanoparticles. Suitable carrier material should be water soluble, and thus is typically prepared from an aqueous solution, which upon drying yields solid carrier material. For aerosol delivery to the lung, the carrier material 34 must be non-toxic and capable of releasing the nanoparticles at the target site, such as by desolving in the aqueous environment of the lung epithelium. In the powder formation step illustrated in FIG. 3, freeze spray drying or bulk drying followed by grinding may be used. According to the method of spray drying, the aerosol particles 38 may be prepared to have a size suitable for delivery to the lung 40 as illustrated in FIG. 4. The aerosol particles 38 deliver the nanoparticles 32 to deep areas of the lung 40. The carrier particles 38 dissolve and release the nanoparticles 32. The nanoparticles deliver the active agent to the target cells. Carrier particles with a size between 1 μm and 5 μm have been found suitable for aerosol delivery to the lung, and hence are respirable, but the deposition of particles is also influenced by inhalation flow rate and other factors. Particles having a size outside of this range may also be respirable depending on for example inhalation flow rates.

Nanoparticles suitable for delivery in the respirable carrier particles may include medical pharmaceuticals and specialties such as preventive agents, for example vaccines, diagnostic agents, for example tracers of various types and imaging enhancers, therapeutic agents, for example drugs, peptides, and radiation, immuno-modulators, vaccine and virus vectors, and combinations of these classes. The nanoparticles may also include respirable non-medical specialties such as physiochemical agents, for example gas antidotes, biophysical modulators, for example paramagnetics, emitters, for example electromagnetic wave emitters, and imaging enhancers.

Gelatin nanoparticles and poly-butylcyanoacrylate nanoparticles were chosen as exemplary materials to demonstrate the effect of the spray-drying process on natural and synthetic carriers. Gelatin is a natural protein made by basic or acetic hydrolysis form collagen. The process influences the pKa of the resulting gelatin. Gelatin A has a pKa of about 8 while gelatin B has a pKa around 5. Due to this property, gelatin particles have a positive or negative surface charge in physiological environments of 7.4 which also can be used for electrostatic drug loading. Protein-based nanoparticles offer a wide range of functional groups which can be used for surface modifications or prodrug synthesis. Carriers made from cyanoacrylate are well characterized in literature. The monomer is known for use to synthesize nanoparticles or nanocapsules. The particles and capsules are biodegradable and biocompatible and have been demonstrated to have cellular uptake in different cell lines.

Carrier particles 38 produced by the method of the preferred embodiment dissolve quickly in aqueous media and release the nanoparticles 32. It is believed on reasonable and probably grounds that smaller nanoparticles will follow in the same mechanism and reach the epithelium cells. Nanoparticles may penetrate the mucosa to enter the interstitial compartment. To avoid macrophage clearance in the lung by alveolar macrophages, protective coatings 26 may be used to modify the particle surface. Previous in vivo studies of intravenous administered nanoparticles have demonstrated that this is a highly efficient way to avoid macrophage clearance by the monocyte macrophage system (MPS).

Example of Powder Formulation

Chemicals—Lactose Monohydrate was obtained from FMC (Philadelphia, USA); Gelatin B from bovine skin (225 Bloom), glutaraldehyde grade I 25% aqueous solution, sulforhodamine 101 acid chloride (Texas Red), fluorescein isothiocyanate-dextran (FITC-Dextran) and cyanoacrylate were obtained from Sigma Chemical (St. Louis, USA); acetone and acetonitrile were purchased from Caleda (Georgetown, Canada). All chemicals were of analytical grade and used as received.

Preparation of Gelatin Nanoparticles

Conventional methods may be used to create nanoparticles, such as the methods of Coester, C. J., Langer, K., Von Briesen, H. and Kreuter, J., Gelatin nanoparticles by two step desolvation—A new preparation method, surface modifications and cell uptake. Journal of Microencapsulation, 17, 187-193 (2000) and Scherer, D., Mooren, F. C., Kinne, R. K. and Kreuter, J., In vitro permeability of PBCA nanoparticles through porcine small intestine. Journal of Drug Targeting, 1, 21-7 (1993). In this example, a two step desolvation method was used to prepare gelatin nanoparticles according to the method described by Coester et al. In brief: 1.25 g of gelatin B was dissolved in 25 mL of distilled water and stirred at 600 rpm and under constant heating of 40° C. 25 mL of acetone were added to the gelatin solution. The high molecular weight (HMW) gelatin precipitated from the solution. The supernatant containing low molecular size gelatin which is still soluble in the aqueous/organic solvent mixture was discarded. The HMW gelatin was re-dissolved in 25 mL of distilled water and stirred at 600 rpm and under constant heating of 40° C.; the pH of the solution was adjusted to 2.5 by adding 1 N HCl; 75 mL of acetone were added to the acidic gelatin solution drop-wise and the nanoparticles precipitated from the solution. 125 μL of 1 mg/mL of solution of Texas Red in acetonitrile was added and stirred for one hour. The particles were stabilized using 400 μL of 25% glutaraldehyde as cross-linking agent and the suspension was left stirring for 12 hours without heating. The remaining solvent was evaporated using a Rotavapor (IKA, Model RV 05, Staufen, Germany); the nanoparticles were purified by centrifugation at 100,000 g (Beckman Model J2-21) for 30 minutes and were washed 3 times with distilled water. The resulting particles were re dispersed in 25 mL of distilled water. The fluorescent-labeled nanoparticles were stored at 4° C. and protected from light.

Gravimetric Determination of the Gelatin Nanoparticles

The nanoparticles were freeze dried using a Labconco (Kansas City) Freeze Dryer model 3 over 24 hours. The particles were completely removed from the bottle and weighed on an analytical balance.

Preparation of Poly Butylcyano Acrylate Particles (PBCA).

Poly butylcyanoacrylate nanoparticles were prepared by an emulsion polymerization process described by Scherer et al. cited above. 50 mg of FITC-Dextran was added to 10 mL of 0.01 N HCl. The solution was stirred at 600 rpm; 100 μL of the monomer were slowly added by pipetter to the solution; the solution was stirred for 4 hours and was protected from light; the pH was subsequently adjusted using 1 N NaOH to pH 5.0. The particles were purified from unbound dye and polymerization residuals as described for the gelatin particles.

Nanoparticles were suspended to 25 mL of distilled water after centrifugation, yielding 2 mg/mL of polycyanoacrylate nanoparticles.

Particle Size Analysis

The particle size of the gelatin and the polycyanoacrylate nanoparticles was determined by photon correlation spectroscopy (Zetasizer model HSA 3000). 100 μL of the nanoparticle suspension were diluted with 4 mL of fresh filtered de-ionized water. The measurements were carried out at room temperature. The particle size was determined before and after spray drying. A 50 mg aliquot of the nanoparticle loaded lactose powder was dissolved in 4 mL of distilled water and the particle size was determined directly without any further dilution.

Spray-Drying of Liquid Containing Nanoparticles

A Mini-Spray Dryer produced by Büchi Laboratoriums-Technik (Flawil, Switzerland) was used. The Mini-Spray Dryer operates on the principle of a nozzle spraying in a parallel-flow in which the sprayed product and the drying air flow in the same direction. The adjustable parameters include inlet and outlet temperature, solution pump flow rate, and the aspirator partial vacuum. In these examples, the inlet air temperatures ranged from 170-180° C., the pump flow rate was 2 mL/min, the aspirator was set to 40 m³/h, and an atomizing air flow rate was 700 L/h (80 psi). A solution containing lactose and nanoparticles was pumped into the feeding system of the spray-dryer. The resultant powder was blown through a cyclone separator and collected in a container. Exhaust air was extracted out of the cyclone by a vacuum pump and filtered by a fiber filter.

To create the mixture of carrier liquid and nanoparticles, 5 g of lactose were dissolved in 75 mL of distilled water and heated up to 40° C. to increase the lactose solubility. The solution was mixed with 25 mL of either gelatin nanoparticles or poly butylcyanoacrylate nanoparticles. The glass chambers of the spray dryer were protected from light. The powders were removed from the collector vessel of the spray dryer and stored at room temperature under light protection.

Fluorescent-Labeling of Lactose

The carrier particles were stained with a florescent label to increase their visibility for the confocal microscopy. The dyes were added to the lactose solution prior to spray drying. The lactose was stained using 500 μL of 1 mg/mL solution of Texas Red in acetonitrile if butylcyanoacrylate particles were used or 500 μL of a 1 mg/mL solution containing 5(6)-carboxyfluorescein if gelatin nanoparticles were used.

Powder Characterization Using Confocal Laser Scanning Microscopy

The morphology of the powders was examined using a Zeiss LSM 510 confocal microscope. The microscope is equipped with the capability to collect 12-bit images using 4 different detectors for fluorescent signals from fluorophores excited by 4 lasers with multiple laser lines (Argon, HeNe1, HeNe2, UV) and a transmission detector for bright field images (DIC). Small aliquots of the spray-dried powders were dispersed in immersion oil on glass slides. The powder particle sizes from all samples were manually measured using the software Metamorph (v. 5.0, Universal Imaging Corporation). At least 25 particles were measured for each powder sample. The mean powder size was calculated based on all measurements.

Powder Dispersion and Sizing by Cascade Impaction

The dispersibility of each powder was assessed using a Mark II Anderson impactor (ThermoAnderson, Smyna, Ga.) with the powder aerosolized using a proprietary, low-resistance dry powder inhaler developed by the Aerosol Research Laboratory of Alberta, Dept. of Mechanical Engineering, University of Alberta, Edmonton, Alberta, Canada. The powder was dispersed at a steady flow rate of 60 L/min. This flow rate was higher than the standard flow rate of 28.3 L/min (1 SCFM) normally used in the Anderson impactor, but was more representative of human inspiratory flow rates in typical dry powder inhalers (DPIs). The Anderson impactor was recalibrated at 60 L/min, using different cut points. Using this calibration the size range of the powder impacted on each plate is known. An inhaler was attached to the inlet of the Anderson impactor and the impactor was fixed on the testing stand horizontally. Using the impactor in a horizontal position does not alter its particle size selection. The flow rate was maintained by a vacuum pump (Emerson Electric Co., USA) and monitored by Pneumotachometer (PT) (4719, Hans Rudolph Inc. 0-100 L/min).

For all of the deagglomeration experiments, the test powder was used in its original state collected from the spray dryer. No post treatment was applied to the powder. The sample powder was weighed using an analytical balance (Sartorius 1207MP2, Germany). Ten powder doses (5 mg each) were loaded individually into the inlet of the DPI. The eight metal plates within the impactor were coated with a thin layer of 316 silicone grease (Dow corning, MI) to prevent fine particles from bouncing on the plates and becoming re-entranced in the air stream, which could give an incorrect size distribution. Indeed, tests done with lower and higher dose loadings did not yield any differences in the measured particle size distributions, indicating an absence of plate overloading. A pre-separator was attached to the top of the impactor to prevent large particles or aggregates from reaching rear stages. Before assembling the apparatus, the inhaler, the pre-separator and all impactor plates were weighed on an analytical balance. After dispersion of the powder into the impactor was completed, the inhaler, the pre-separator, and all the impactor plates were weighed again by the same balance. The fine particle fraction (FPF_{ED<5.6 μm}), or equivalently the respirable fraction, was determined by the weight increase of each part. For accuracy, each test was repeated three times.

Statistical analysis—A paired t-test was performed to compare the sizes of nanoparticles before spray-drying into powders and after release from dissolved powders, and another to compare the size of powders with and without nanoparticles at the statistical P-value of 0.05.

### Results

The mean particle size of the nanoparticles was 242 nm±14 nm for gelatin and 173 nm±63 nm for butylcyanoacrylate. The gravimetric determination of the gelatin nanoparticles after freeze drying revealed that 69±5.3% of the initial amount of gelatin formed nanoparticles. The spray-dried lactose produced spherical powders as illustrated in FIGS. 5A, 5B and 5C. CLSM cross-sections through the powders showed that some particles were hollow while other powder particles had a continuous matrix. A continuous matrix is illustrated in FIGS. 5A and 5C, and the contained nanoparticles are shown in FIGS. 5A and 5B. Both Texas Red and 5(6)-carboxyfluorescein stained lactose very well. The mean particle size determined by CLMS measurements of pure lactose powders, powders with gelatin nanoparticles and powders with polycyanoacrylate nanoparticles were 2.50, 2.59 and 2.60 μm, respectively. A t-test was performed to compare the size of these different powder types and showed that the incorporation of nanoparticles into lactose by spray-drying did not affect the size of the powders formed.

Gelatin Nanoparticle Loaded Lactose Powders

Gelatin nanoparticles stained with Texas Red were incorporated into lactose powders by spray-drying (see FIGS. 5A, 5B and 5C). As seen, the distribution of gelatin nanoparticles was even throughout the carrier particle. CLSM cross-section images through the carrier particle were taken to further examine the distribution (figure not shown). It was observed that gelatin nanoparticles do embed in the body of the carrier. In some instances clusters of gelatin nanoparticles were observed as bigger red spots within the particles or they appeared as polarized red staining on one side of the particles (figures not shown).

Polycyanoacrylate Nanoparticles Loaded Lactose Powders

FITC-Dextran stained polycyanoacrylate nanoparticles in lactose carrier particles formed a hollow carrier particle. Cross-section images show that the butylcyanoacrylate particles tend to accumulate more as clusters within the carrier particle compared to the gelatin nanoparticles. Larger lactose particles tend to contain more clusters of the polycyanoacrylate nanoparticles than smaller ones. Visual observation showed a continuous distribution of the nanoparticles between different particle sizes.

Powder Dispersion Using a Dry Powder Inhaler

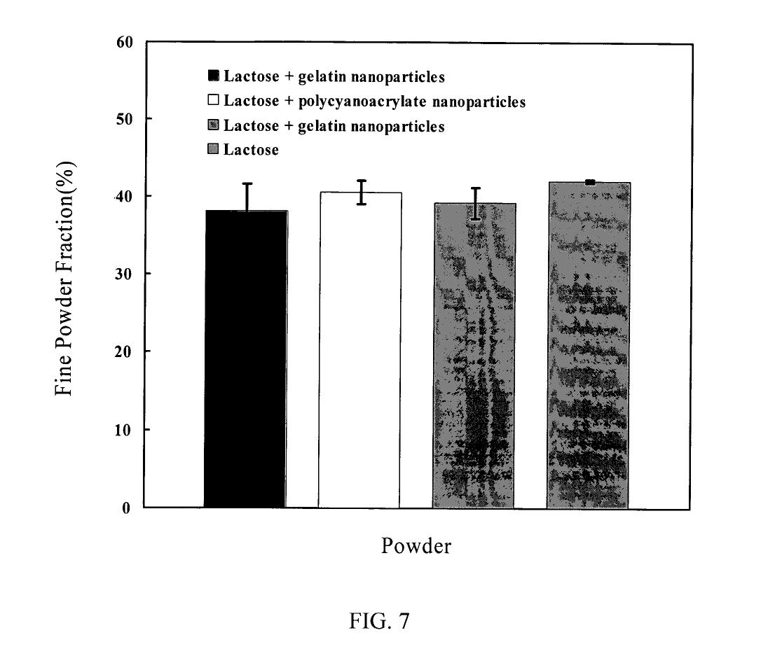

The powder recovery from the cascade impaction test was >90%. FIG. 7 shows all particles smaller than 5.6 μm. This represents the fine particle fraction (FPF_{ED<5.6 μm}). Three batches of four typical powder formulations were deagglomerated by the same inhaler. The results indicate that the FPF_{ED<5.6 μm} varied within a narrow range of 38%-42%. The error bars represent the standard error. The presence of the nanoparticles had no significant effect on the fine particle fraction of the powders.

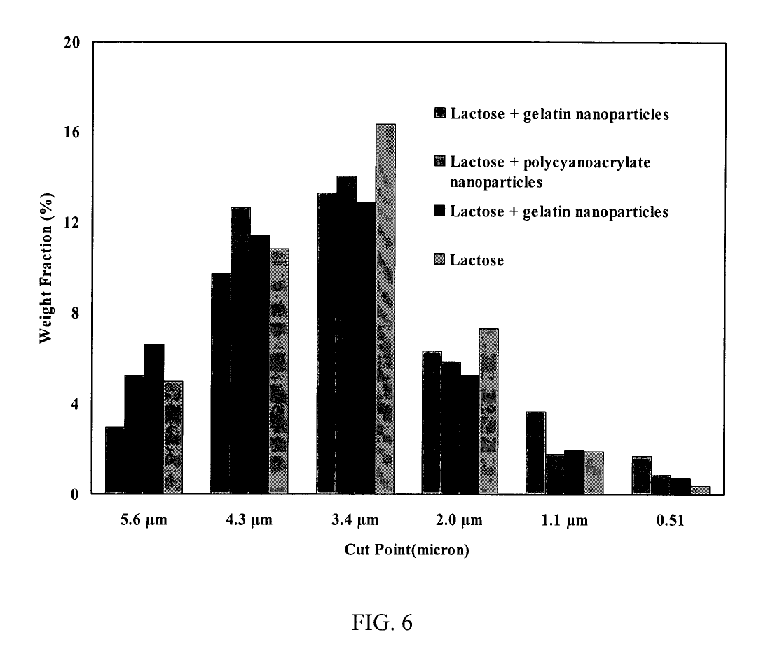

FIG. 6 shows the size distribution of the aerosolized particles. Each group represents the powder size on a defined stage of the cascade impactor. The powder deposition increased from stage 1 to stage 3 (5.6 μm down to 3.4 μm) and decreased from stage 3 to stage 6 (3.4 μm to down to 0.5 μm). About 16% of the dose was collected in sizes larger than 3.4 μm, while 15% of the carrier particles were collected on the 3.4 μm stage. The MMAD of the powders was 3.51 μm. The comparison of the data indicates that no statistical significant differences occur on the aerodynamic diameter distribution among the different lactose powder types (t-test; P=0.05).

Effect of Spray-Drying on the Size of Polycyanoacrylate Nanoparticles and Gelatin Nanoparticles

The mean particle sizes of the gelatin and poly butylcyanoacrylate nanoparticles were measured before spray drying and after re-dissolving of the spray-dried powders. The mean particle size of gelatin nanoparticles increased from 242.2±17 nm to 319.9 nm±58. The average size of the poly butylcyanoacrylate particles was 173.0±59 nm before spray drying and 231.7 nm±33 nm after spray drying. A t-test was performed to compare the size of individual nanoparticle type before and after spray-drying at P=0.05. Although after spray-drying the gelatin nanoparticles were still in the nano-range, they differ significantly in size from the original; whereas the difference between poly-butylcyanoacrylate nanoparticles before and after spray-drying was statistically insignificant.

Visualizing different layers of the carrier particle using CLMS has shown that the nanoparticles are homogeneously distributed throughout the matrix of the particle (figures not shown). The larger size of the gelatin nanoparticles after spray-drying may be a result of a change in conformation under the thermal condition of spray drying that might be overcome by a lower thermal exposure of the spray drying process or using spray freeze dying. The latter process is preferable if heat-sensitive drugs are attached to the gelatin nanoparticles. However, studies have shown that suitable spray-drying conditions expose biological molecules only for milliseconds to seconds in the spray dryer chamber and it has been argued that this might not cause extensive damage given that the powder temperature is in the order of 40-45° C.

In some carrier particles clusters of gelatin nanoparticles were observed. Such clusters, if not deaggregated after the carrier particle dissolves, may also cause an increase in the particle size. The tendency of proteins and peptides to accumulate on the surface of spray-dried powders as clusters has been described in various studies. During atomization, the liquid/air interface of the spray solution greatly and suddenly expands. This is a distinct interface in which proteins or peptides tend to adsorb to each other and the gelatin nanoparticles in this study are no exception. However, prior studies have shown that it is known that adding polysorbate 20 to the spray drying process reduced surface aggregation of recombinant human growth hormone by 15% to <2%. In another study using bovine serum albumin (BSA), similar results were reported in which increasing concentrations of polysorbate 80 or sodium dodecyl sulfate reduced the surface accumulation of BSA in a concentration-dependent manner.

In hollow carrier particles, formation of nanoparticle clusters may be due to the adhesive nature of cyanoacrylate nanoparticles or their free surface energy. However, dissolving the particles in water revealed that the clusters do not stick together. The particle size analysis shows that the spray-drying process has no effect on the average size of the nanoparticles. This might be due to the dissolution process of the carrier particles which contributes to the deagglomeration of the nanoparticle clusters.

Aerosol powders ranging from 1 to 5 μm are considered the optimum size for deposition beyond the increasingly narrow airways into the alveoli. However, such particles often also stick together which lowers the fine particle fraction. One approach known in the art to overcome this is the use of large porous particles (>5 μm) with a low mass density (<0.4 g/cm³). It has been shown that larger particles aggregate less and deaggregate more easily. Another approach is the use of high efficiency dry powder inhalers. Such powder inhalers are able to deagglomerate powders more ably than conventional powder inhalers. The results of our cascade impactor tests clearly show that the spray-died powders can be aerosolized and a high percentage is in the fine particle range appropriate for inhalation.

The described delivery technology can be used for lung specific applications such as lung cancer, cystic fibrosis or asthma. However, patients with systemic diseases can also benefit from such delivery technology as nanoparticles facilitate the entry of drugs and proteins through the lung epithelium into the systemic circulation.

Nanoparticle-loaded carrier particles produced by the preferred embodiment of spray drying are different from the particles described in the study of Tsapis, N., Bennett, D., Jackson, B., Weitz, D. A. and Edwards, D. A., Trojan particles: Large porous carriers of nanoparticles for drug delivery. Proceedings of the National Academy of Sciences of the United States of America, 99, 12001-12005 (2002). The cited study used large porous particles for potential pulmonary nanoparticle delivery and found that the concentration and the nature of the nanoparticles determined the shape and the size of the resulting aerosol particles. In contrast, the shape and size distribution of the carrier particles described here are independent of the presence of the nanoparticles.

The present disclosure demonstrates incorporation of nanoparticles into respirable carrier particles. The described carrier particles can deliver nanoparticles into the lung. The size and shape of the spray-dried powders is suitable for respiratory deposition of the carrier particles. The carrier is expected to dissolve quickly after landing on the aqueous covered epithelium of the lung. In vitro results show that the delivered nanoparticles are released instantly. Nanoparticles can be loaded with active principles like drugs, peptides, oligonucleotides or diagnostics for local or systemic delivery of the active principles. This delivery platform opens up a wide range of treatment applications of pulmonary and systemic diseases using targeted delivery strategies via nanoparticles.

A person skilled in the art could make immaterial modifications to the invention described in this patent document without departing from the invention.

1. A method of formulating a powder containing nanoparticles for aerosol delivery to the lung, the method comprising the steps of:
mixing nanoparticles with a liquid carrier to create a mixture; and
forming the mixture into carrier particles having a size suitable for aerosol delivery to the lung.

2. The method of claim 1 in which the mixture is formed into carrier particles by drying followed by breaking up the dried mixture.

3. The method of claim 1 in which the mixture is formed into carrier particles by spray drying.

4. The method of claim 1 in which the mixture is formed into carrier particles by freeze spray drying.

5. The method of claim 1 in which the nanoparticles contain an active agent selected from the group consisting of therapeutic, diagnostic and preventative agents.

6. The method of claim 1 in which the carrier particles have a mass median aerodynamic diameter between 1 μm and 5 μm.

7. A method of treating a human being, the method comprising the steps of:
forming a carrier particle by the method steps of claim 1, in which the nanoparticles contain an active agent; and
delivering the carrier particle to the lung of the human being by aerosol delivery.

8. The method of claim 7 in which the active agent is selected from the group consisting of therapeutic, diagnostic and preventative agents.

9. The method of claim 8 in which the active agent is a therapeutic agent.

10. The method of claim 8 in which the active agent is a diagnostic agent.

11. The method of claim 8 in which the active agent is a preventative agent

12. The method of claim 8 in which the active agent is radiation.

13. The method of claim 8 in which the active agent is selected from the group consisting of immuno-modulator and vaccine.
